(19) United States
(12) Patent Application Publication
(10) Pub. No.: US 2017/0273618 A1
(43) Pub. Date: Sep. 28, 2017

(54) USE OF DEXA SCANS TO ASSESS BODY FAT OUTSIDE OF HEALTHCARE SETTINGS

(71) Applicants: John Fullerton, Fairfax, CA (US); Kate McElroy, Fairfax, CA (US)

(72) Inventors: John Fullerton, Fairfax, CA (US); Kate McElroy, Fairfax, CA (US)

(21) Appl. No.: 15/504,923

(22) PCT Filed: Aug. 17, 2015
(86) PCT No.: PCT/US15/45564

§ 371 Date: Feb. 17, 2017

Related U.S. Application Data
(60) Provisional application No. 62/038,759, filed on Aug. 18, 2014.

(51) Int. Cl. A61B 5/00; G06F 19/00; A61B 5/08; A61B 6/00; A61B 5/02

(52) U.S. Cl. CPC A61B 5/4872; A61B 5/4509; A61B 6/482; A61B 6/505; A61B 6/563; A61B 5/02; A61B 5/08; G06F 19/328; G06F 19/3481; G06F 19/3475

(57) ABSTRACT

The present application describes fitness facilities that include DEXA scanners, and methods of use thereof to establish or modify fitness and/or nutritional regimens for patrons of the fitness facilities. Various fitness facilities are described in which the interpretation of DEXA scan results is performed either on- or off-site. The fitness facilities of the invention can include a counseling center staffed with fitness trainers and/or nutrition experts for counseling subjects on issues relating to fitness and/or nutrition.

### CROSS REFERENCE TO RELATED APPLICATIONS

This application claims priority under 35 U.S.C. §119(e) from U.S. Provisional Application No. 62/038,759 filed on Aug. 18, 2014, which is incorporated herein by reference in its entirety.

### FIELD OF THE INVENTION

The present invention generally relates to systems and methods for evaluating and improving the physical health of individuals.

### BACKGROUND

The following description includes information that may be useful in understanding the present invention. It is not an admission that any of the information provided herein is prior art, or relevant to the presently claimed invention.

Body fat percentage is an important biomarker for numerous medical conditions. High body fat percentage is associated with conditions including but not limited to high blood pressure, diabetes, heart disease, cancer, infertility, back pain, skin infections, gastric ulcers, and gallstones. On the other hand when body fat percentage is too low, it is associated with increased susceptibility to fractures, infertility, various intestinal diseases and various neurological conditions. Considering the implications of having a body fat percentage that is either too low, or too high, routine testing can be a very helpful tool for guiding the maintenance and improvement of health. Unfortunately, body fat percentage testing is vastly underutilized, partly due to the drawbacks associated with traditional testing—from inaccuracy to embarrassment. There is clearly a need in the art for systems and methods for offering more accurate, discreet, and accessible body fat testing.

### SUMMARY OF THE INVENTION

In various embodiments, the invention teaches a method for delivering body fat percentage analysis and/or bone density analysis to a subject at a fitness facility. In some embodiments, the method includes scanning the subject's body by utilizing a dual-energy X-ray absorptiometry (DEXA) scanner located at the fitness facility; obtaining results regarding the subject's body fat percentage and/or bone density based on the DEXA scan; and providing results to the subject regarding the subject's body fat percentage and/or bone density. In some embodiments, the results of the DEXA scan are determined at the fitness facility. In certain embodiments, the results of the DEXA scan are determined outside of the fitness facility. In some embodiments, the fitness facility is staffed with one or more staff member trained to read and interpret the results of the DEXA scan. In certain embodiments, the fitness facility includes one or more device for strength training and/or aerobic training. In some embodiments, the fitness facility is staffed with one or more staff member trained to design or modify a fitness and/or nutrition and/or rehabilitation regimen for the subject at the fitness facility based partly upon the results of the DEXA scan. In certain embodiments, the fitness facility is staffed with an expert in nutrition selected from the group consisting of a nutritionist, a dietician, and a registered nurse. In some embodiments, the fitness facility includes a swimming pool and/or a spa and/or a sauna. In certain embodiments, the fitness facility is staffed with one or more certified personal trainer. In some embodiments, the fitness facility is staffed with one or more medical professional selected from the group consisting of a physician, a nurse, and a dietician. In some embodiments, the fitness facility includes at least one device configured to assess cardiovascular health. In some embodiments, the fitness facility is staffed with one or more staff member trained to utilize the device configured to assess cardiovascular health. In certain embodiments, the fitness facility is staffed with one or more staff member certified to conduct plastic surgery. In some embodiments, the fitness facility includes one or more device configured to assess pulmonary health. In certain embodiments, the fitness facility is staffed with one or more staff member trained to utilize the device configured to assess pulmonary health. In some embodiments, the fitness facility is staffed with one or more physical therapist. In certain embodiments, the fitness facility is staffed with one or more staff member trained in improving or maintaining the physical and/or mental health of a subject who is from 50-100 years old. In some embodiments, the subject is from 50-100 years old. In certain embodiments, the subject has been referred to the fitness facility by a medical professional for the purpose of recovering from an injury or illness.

In various embodiments, the invention teaches a method for generating revenue for a fitness facility. In certain embodiments, the method includes: performing services comprising dual-energy X-ray absorptiometry (DEXA) scanning and providing the results thereof to a subject at the fitness facility; and collecting a fee from said subject for performing said services. In certain embodiments, the fee for performing said services is charged separately from a periodic membership fee paid by the subject for the use of the fitness facility.

In certain embodiments, the invention teaches a method for determining and/or modifying an exercise and/or nutritional regimen for a subject at a fitness facility. In some embodiments, the method includes utilizing a dual-energy X-ray absorptiometry (DEXA) scanner located at the fitness facility to perform a DEXA scan of the subject in order to determine bone density and/or body fat percentage of the subject; and determining and/or modifying an exercise and/or nutritional regimen for the subject, based at least partly upon the results of said DEXA scan. In certain embodiments, the exercise and/or nutritional regimen for the subject is determined and/or modified by a personal trainer and/or a nutrition expert who are staff members of the fitness facility. In some embodiments, the fitness facility includes a nutrition center that offers for sale one or more foods and/or nutritional supplements. In certain embodiments, one or more of the foods and/or nutritional supplements are through or known to improve bone density alone and/or in conjunction with strength training. In some embodiments, one or more of the nutritional supplements is intended for consumption by a subject who is from 50-100 years of age. In some embodiments, the fitness facility offers cosmetic products for sale.

### BRIEF DESCRIPTION OF THE DRAWINGS

Exemplary embodiments are illustrated in the referenced figures. It is intended that the embodiments and figures disclosed herein are to be considered illustrative rather than restrictive.

FIG. 1 depicts, in accordance with an embodiment of the invention, a diagram of a fitness facility environment 100. The fitness facility environment 100 includes fitness equipment 101, and a dual-energy X-ray absorptiometry (DEXA) scanner 102 for performing DEXA scans on subjects/patrons of the fitness facility 100. The fitness facility environment may also optionally contain a health and wellness counseling center 103 where certified personal trainers and/or nutritionists and/or counselors counsel subjects/patrons of the fitness facility regarding weight loss, fitness, and other health-related issues. The fitness facility environment 100 optionally includes a nutrition center 104, where various foods and supplements are sold, and where a person skilled in nutrition may counsel subjects/patrons of the fitness facility 100.

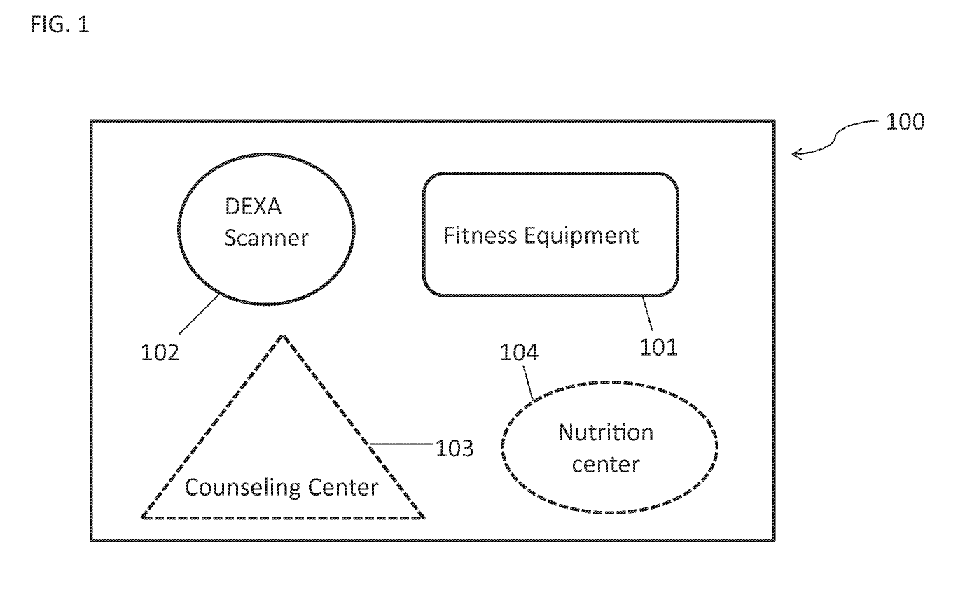

FIG. 2 depicts, in accordance with an embodiment of the invention, a flow chart demonstrating DEXA scanning of a subject/patron of a fitness facility and subsequent interpretation of scan results, either on-site (Path A) or off-site (Path B) and communication of scan results to the subject/patron.

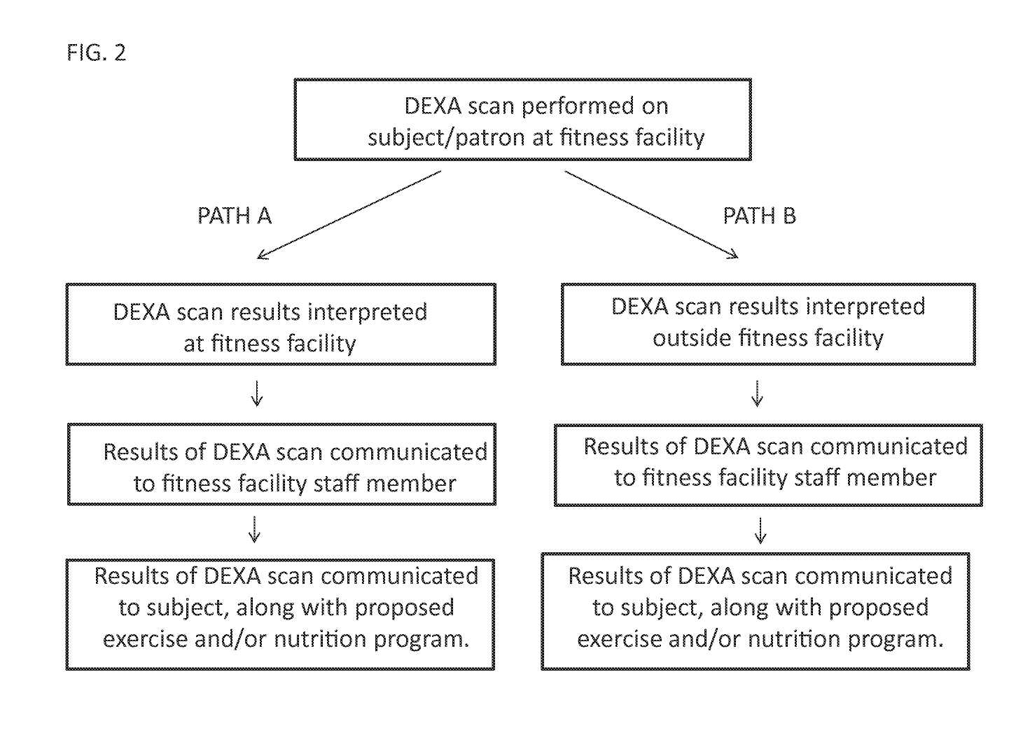

FIG. 3 depicts, in accordance with an embodiment of the invention, a fitness facility environment 200 which includes (1) a DEXA Scan Center 201 (which includes a DEXA scanner 206 and a DEXA technologist for administering a DEXA scan), (2) fitness equipment 202, (3) a Counseling Center 203 staffed with staff members for counseling subjects/patrons regarding health and fitness issues, (4) a Computer System 204 for storing and accessing data relating to subjects/patrons, which optionally includes results from one or more DEXA scan. The computer system 204 is optionally accessible by one or more staff member of the Counseling Center 203 and/or DEXA Scan Center 201 and/or Off-site Physician 205.

FIG. 4 depicts, in accordance with an embodiment of the invention, a fitness facility environment 300, which includes a DEXA scanner 301 for body fat testing and/or bone density testing in a gym environment that includes fitness equipment 304. The DEXA scanner 301 can optionally be incorporated in an ambulatory care clinic 302. Overall, the fitness facility environment 300 can optionally include one or more of the following: (1) an ambulatory care clinic 302, (2) a counseling center 303, (3) a computer system 305, (4) a juice bar 306, (5) a nutrition center 307, (6) a restaurant 308, and (7) a teaching facility 309. The optional computer system 305 of the fitness facility 300 can be optionally configured to receive information from and send information to a computer device located at or within one or more of the DEXA scanner 301, ambulatory care clinic 302, counseling center 303, fitness equipment 304, juice bar 306, nutrition center 307, restaurant 308, and teaching facility 309. The computer system 305 can be further optionally configured to receive information from and send information to a computing device utilized by an off-site physician 404. By including one or more of the optional components demonstrated in fitness facility 300 (and optionally by using an off-site physician 400), an environment conducive to improving the mental and/or physical health of patrons/individuals can be established.

FIG. 5 depicts, in accordance with an embodiment of the invention, a diagram of a hardware environment and an operating environment in which the components and the computing devices of FIGS. 3 and 4 may be implemented. Merely by way of example, the computer system depicted, or one or more components thereof, can be implemented in environment 300 (computer system 305) or environment 200 (computer system 204) to facilitate operation of a fitness facility and communication of DEXA scan data and results.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs.

One skilled in the art will recognize many methods and materials similar or equivalent to those described herein, which could be used in the practice of the present invention. Indeed, the present invention is in no way limited to the methods and materials described.

By way of additional background, historically, in the gym/spa environment, body fat percentage has been an important quantitative bio-marker to assess and to follow for athletes and non-athletes alike. The most widely available types of testing currently available include skin fold measurement, bioelectric impedance, anthropometric, and hydrostatic weighing. Each of these traditional methods has one or more suboptimal characteristics that contribute to underuse, especially at fitness facilities.

Skin fold testing involves pinching and measuring skin folds with calipers at specific locations in a subject's body. Unfortunately, the accuracy of this technique is highly dependent upon the skill level of the tester in properly selecting, pinching, and measuring skin within relatively narrow regions of a subject's body. The results of skin fold testing can also vary greatly depending upon which of several formulas are used, and how many sites are tested.

Bioelectric impedance analysis, or BIA, determines the electrical impedance, or opposition to the flow of an electric current through the body. Muscle has high water content, and is highly conductive, while fat has lower water content and is not as highly conductive. Based upon the strength of the impedance, along with height and weight metrics, a BIA scale, or handheld device will estimate fat-free body mass and body fat percentage. Accuracy of BIA, however, is significantly influenced by the hydration level of the subject to be tested, and the region through which electrical current is administered (i.e through the upper or lower body).

The anthropometric method requires using body circumference measurements (typically waist and neck for men and hips and neck for women), along with height and weight metrics, to estimate body fat percentage. Because body fat is not directly measured, this method only provides a very rough estimate of body fat percentage.

Hydrostatic testing requires submerging a subject in a specialized tank of water. Because bone and muscle are denser than water, a subject with a larger percentage of fat free mass will weigh more in the water and have a lower body fat percentage. Conversely, a large amount of fat mass will make the body lighter in water. Although hydrostatic testing is generally considered to be relatively reliable, accuracy is partly contingent upon the consistency in the process of blowing as much air as possible out of the lungs during pretest screening. Hydrostatic testing is also problematic because it requires wearing a bathing suit, and it is often performed in an environment that is relatively open to viewing by others, which is considered by many to be an embarrassing process.

In contrast with the aforementioned methods of body fat percentage testing, dual-energy X-ray absorptiometry (DEXA) is highly accurate, can be performed discreetly, and offers the added benefit of accurately measuring bone density. In fact, medical facilities (including ambulatory/out-patient clinics) have propounded the use of DEXA (e.g. Bone Mineral Density-BMD) scans to measure the “tensile strength” regarding mineralization, particularly at strategic sites for long-term weight-bearing capability (and therefore fracture potential) in the curvature of the lower (lumbo-sacral) spine, both femoral necks of the hips, and the radial articulation at both wrist sites. In most people this mineralization/strength at these sites “peaks” in their thirties and then gradually falls off and diminishes, causing lower mineralization (e.g. osteopenia) or the disease of pathologically weakened bones (called osteoporosis)—depending upon genetics, Asian descent, age, sex, cigarette smoking, hormonal influences (including both low estrogen and/or low testosterone), amount and frequency of weight-bearing exercise, diet, supplementation, the use of certain medications (e.g. prednisone) and other factors.

Considering the advantages of DEXA scanning, there are numerous benefits to incorporating a DEXA scanner into fitness facilities, especially those focused on providing a more comprehensive approach to fitness and health. As described in greater detail herein below, in some embodiments the fitness facilities of the invention in which DEXA scanners are incorporated provide family oriented and socially desirable environments, and resources for performing a wide-range of healthy activities geared toward patrons of almost all ages. Advantageously, the fitness facilities described herein can optionally offer one or more diagnostic health services, therapeutic health services, preventative care services, mental health services, and/or other health services. These services can cause members (individuals and families) of the facilities described herein to be more targeted toward achieving their respective health and fitness goals, and more prone to attend and spend time and resources at the facilities. The various fitness facility environments described herein allow/encourage patrons (individuals and families) to take more responsibility for their physical and mental health, preventive medical care and screening, and general lifestyle. With the above considerations in mind, additional specific embodiments of the invention are provided herein below.

In various embodiments, the invention teaches providing a DEXA scanner within a fitness facility, so that body fat percentage analysis and/or bone density analysis of a subject/patron can be performed on-site at said facility. Although numerous types of DEXA scanners could be used, in certain non-limiting examples, the DEXA scanner included in the fitness facility can include any full table DEXA scanner that uses a fan-beam source and one or more detectors. In certain preferred embodiments, the DEXA scanner is a hospital grade 18 channel (or higher) General Electric Dexa Scanner.

The information obtained from a DEXA scan regarding a subject's body fat percentage and/or bone density can be used to facilitate the development of an appropriate exercise and/or nutrition regimen for the subject. Merely by way of example, body fat percentage and/or bone density determined from a DEXA scan can be used to establish a starting point for a subject beginning an exercise routine. A fitness trainer, and/or nutritionist and/or other qualified individual described herein, may then determine an appropriate exercise routine with a goal toward the subject achieving a specific body fat percentage and/or bone density level over time. Merely by way of example, the fitness trainer and/or nutritionist and/or other qualified individual described herein may together or individually determine a healthy daily, weekly or monthly caloric deficit by way of exercise and/or diet that will lead to a desired amount of weight loss, and attendant body fat percentage reduction for the subject over a certain period of time. In alternative embodiments, a fitness trainer and/or nutritionist and/or other qualified individual described herein may together or individually devise or alter an exercise and/or nutrition regimen in order to increase a subject's body fat percentage, using diet and/or exercise, because of the results of a DEXA scan. In certain embodiments, a fitness facility patron's body fat percentage may be serially tested, and the patron's progress or lack of progress may thus be monitored, leading to altering or maintain the subject's diet or exercise program, as appropriate. In certain embodiments, if a subject's/patron's bone density is determined to be low, based upon the DEXA scan, a fitness trainer or other qualified staff member may devise or alter an exercise and/or nutrition regimen in order to increase a subject's bone density (e.g. by incorporating weight training into the subject's exercise routine and/or devising a diet plan, with or without nutritional supplements).

A fitness facility of the invention, at which DEXA scanning is to be implemented, could be configured in any of a number of ways, and thus is not limited to one particular embodiment. In fact, the term “fitness facility,” as used herein, is intended to include any fitness facility at which a subject can perform exercise of any kind. In some embodiments, the fitness facility can include, but is in no way limited to, one or more free weights and/or strength training machines. In certain embodiments, the fitness facility can include space and equipment for performing one or more types of exercise that includes, but is in no way limited to, yoga, dancing, martial arts, stretching, strength training, aerobic training, basketball, baseball, volleyball, soccer, football, water aerobics, swimming, and golf. In addition to the many possible types of equipment that could be included in the fitness facility, the fitness facility can also be staffed in many different ways. In certain embodiments, the staff members of the fitness facility can include, but are in no way limited to, one or more personal trainer, coach, person trained to perform a DEXA scan, person trained to read a DEXA scan, nutritionist, dietician, life coach, psychologist, psychiatrist, physical therapist, exercise physiologist, massage therapist, acupuncturist, physician, physician's assistant, nurse, salesperson, cook, receptionist, combinations thereof and the like.

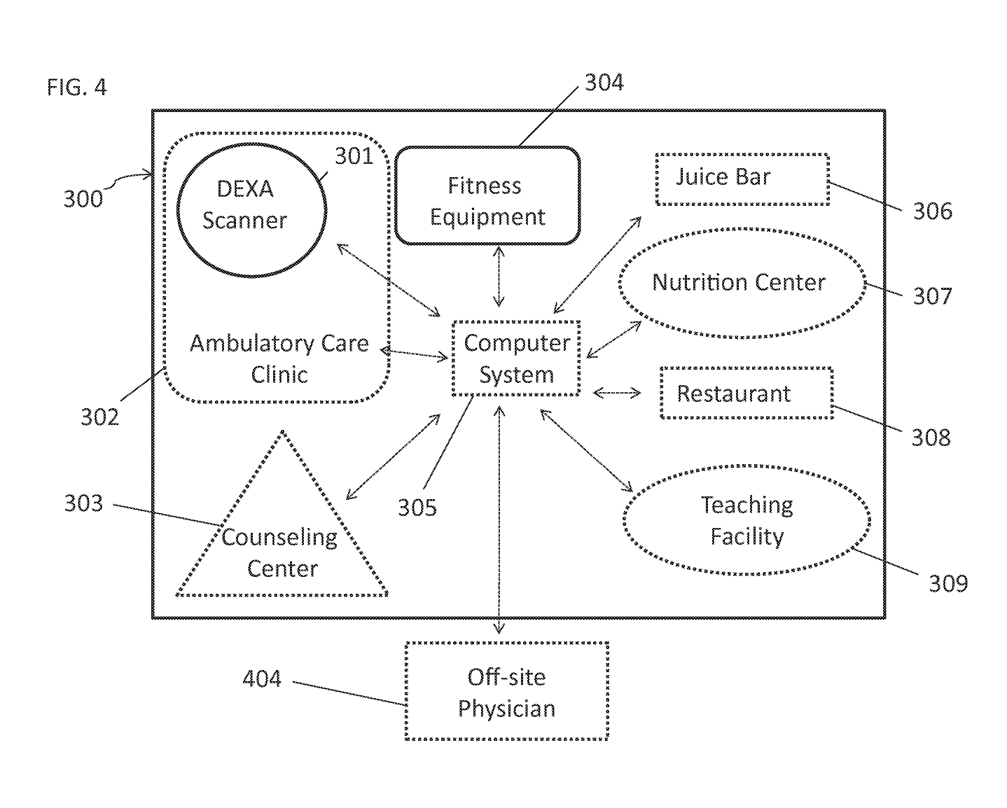

In preferred embodiments, the staff members of the fitness facility regularly communicate and thereby coordinate the training and/or treatment of fitness facility patrons by attending meetings held daily, weekly, or monthly. Advantageously, coordinating and tracking training and/or treatment, including scheduling of the patrons for testing and counseling, can be accomplished using a computer system that includes a database for storing patron information (e.g. DEXA testing results, various fitness metrics, training programs, attendance, notes, contact information, foods and/or beverages and/or nutritional supplements and/or prescription medications consumed and/or purchased, information obtained from any of the components of the facility described in FIG. 4, and the like). In preferred embodiments, the patron information in the computer system can be accessed by staff of the fitness facility by using one or more password associated with a staff member's profile. In some embodiments, subjects/patrons may access their own information stored in the computer system, at various terminals in the facility and/or via wireless web access using a unique password.

In certain embodiments, in addition to exercise equipment and space for performing specific exercises, the fitness facility can include a nutrition center configured for the sale of nutritional supplements and/or foods and/or beverages of various kinds. In certain embodiments, the nutritional supplements can include those that are known or believed to increase bone density, alone or in conjunction with weight bearing exercise. Merely by way of non-limiting examples, these supplements can include ingredients that can include, but are in no way limited to, black cohosh, black tea, calcium, copper, DHEA, equol, evening primrose oil, fish oil, flaxseed, fluoride, green tea, horny goat weed, ipriflavone, magnesium, manganese, mate, oolong tea, red clover, silicon, soy, strontium, vitamin C, vitamin D, vitamin K, zinc, combinations thereof, and the like. In some embodiments, the nutrition center can additionally or alternatively offer for sale one or more nutritional supplements and/or foods and/or beverages known or thought to be useful for gaining or losing weight, maintaining a healthy weight, or maintaining or restoring healthy electrolyte levels. These items can include, but are in no way limited to vitamins such as omega 3 fatty acids, chromium piccolinate, low carbohydrate drink supplements, protein supplements (beverages and foods), and fluid/electrolyte replacement formulae.

In some embodiments, the nutritional supplements described herein can be sold at a juice bar located at the fitness facility. In various embodiments, the juice bar can be (a) located adjacent to the nutrition center, (b) included within the nutrition center, or (c) separated from the nutrition center.

The fitness facility can offer for sale one or more pharmaceutical products/programs known to be useful for losing weight, including, but in no way limited to Phentermine-based, Meredia-based, and Orlistat-based programs, along with other evidence-based prescription medications. These medications can be prescribed by physicians, on- or off-site and used in conjunction with or separately from the neutraceuticals described herein. Pharmaceutical products can be offered for sale in the nutrition center, or they can be offered for sale elsewhere within the fitness facility. Merely by way of example, the fitness facility can include a pharmacy where pharmaceutical products are offered for sale. In each case, prescription medications available at the fitness facility are always prepared and/or dispensed and/or sold by a person legally authorized to do so.

In certain embodiments, the fitness facility can include one or more restaurants. In some embodiments, the one or more restaurants offer cooking classes.

In certain embodiments, the fitness facility includes a teaching facility (e.g. the teaching facility 309 in FIG. 4). Merely by way of non-limiting examples, the teaching facility can offer classes related to weight management, nutrition, cooking, aging, exercise, preventative health care, mental health, marriage counseling, sexual education, self-esteem, and the like.

In some embodiments, the fitness facility offers practical training for DEXA scan technicians to operate a DEXA scanner in conjunction with a certification program or continuing education. In certain embodiments, specific clients of the facility with particular physiological conditions, or suspected conditions, may be selected to train the DEXA scan technicians.

In some embodiments, the fitness facility offers training for the certification or continuing education of a personal trainer. In certain embodiments, the training includes teaching a personal trainer to utilize the results of a DEXA scan to establish an appropriate fitness and/or diet program for a subject/patron of the fitness facility. Merely by way of example, a personal trainer may be taught that if low bone density is determined by a DEXA scan, then a fitness program that gradually implements weight bearing exercise, and/or a diet program geared towards improving bone density should be recommended.

Importantly, the various possible weight management and/or sports training and/or fitness training approaches offered to the patrons/individuals can be developed and/or monitored not only by personal trainers and/or exercise physiologists at the facility, but also (or alternatively) by life coaches, health coaches, diabetic educators, and preventive medicine/lifestyle doctors (and specialists). Appropriately qualified staff members of the med/spa/gym/facilities described herein can employ lifestyle motivational interviewing techniques along with the aforementioned prescription medications and/or nutraceuticals and/or exercise programs to advance the health of the individuals/patrons of the facilities described herein.

In various embodiments, the fitness facility is optionally configured to include staff members, equipment, dressings and medicaments for performing one or more cosmetic procedures. These procedures can include, but are in no way limited to A. wrinkle treatments with Botox and/or high frequency ultrasound; B. treatments for repairing sun damage; C. treatments for reducing redness/facial pigmentation; D. treatments for reducing unwanted hair or treating unwanted hair loss; E. non-surgical body sculpting/lifting; F. body contouring, flattening of the abdomen, and generally sculpting areas of the body with liposuction, and the like; G. lengthening eyelashes; H. body rejuvenating/relieving stress with massage, alone or in conjunction with yoga, etc.; I. reducing excessive sweating (hyperhydrosis) with Botox; J. utilizing hormone replacement therapies; K. prescription and/or non-prescription acne/acne scar treatment; L. facials/exfoliations/dermabrasions, and the like.

In some embodiments, the fitness facility is configured with numerous private rooms such that certain services offered at the fitness facility, and especially those for which privacy may be preferred, can be performed privately (e.g. massage rooms, acupuncture rooms, DEXA testing rooms, life coaching rooms, therapy (physical or mental) rooms, rooms for medical evaluation or treatment, dietary coaching rooms, etc.).

In various embodiments, the fitness facility of the invention includes an evaluation/consulting office on-site where “healthy” individuals between the ages of 15-118 years old may be specifically targeted more effectively for preventive medical practices by emphasizing the primary goal (and procedure result) of body fat composition/fitness and secondarily “maintaining” healthy bones via the secondary DEXA BMD test results that globally serves to augment key markers of metabolic and end-organ (bones) health and fitness and awareness via life/health coaching. In some embodiments, the individuals can be 15-118, or 20-100, or 30-80, or 40-70, or 50-60 years old. In some embodiments, the individuals are recovering from an illness or injury.

In some embodiments, pulmonary and cardiac testing and follow-ups may likewise be best coordinated by this innovative approach to having one or more evaluation/consulting offices on-site. In certain embodiments, the fitness facility includes an ambulatory care clinic, at which individuals/patrons may be assisted by skilled medical professionals (as described herein) for the purposes of preventing, diagnosing and/or prognosing and/or treating illnesses and/or injuries. In some embodiments, the fitness facility is configured with equipment and staff members necessary to perform tests/evaluations including but in no way limited to evaluating vital signs, O₂ saturations (rest and exercise), spirometry, EKG's, holter monitor testing (short- or long-term), ECHO's/ultrasounds, stress tests, stress ECHOS, and the like.

The diagnosis of osteoporosis, obesity, and/or any other medical condition, described herein or otherwise, can be kept in the subject/patron's medical record on-site or communicated to the subject's primary care physician or relevant specialist, in accordance with applicable Health Insurance Portability and Accountability Act of 1996 (HIPAA) laws and regulations. It is intended that staff for this aspect of the invention could include one or more properly licensed and trained (and retrained) service providers, including but in no way limited to a licensed reading radiologist, a DEXA procedure technologist, a life coach who specializes in bone health and weight management counseling, and the like. In some embodiments, some or all of the aforementioned staff members are located on-site. In other embodiments, some or all of the aforementioned staff members are located off-site, and only visit for scheduled appointments. In some embodiments, any medical condition detected by the DEXA scan, or any other form of testing described herein, is reported electronically to the subject's/patron's primary care physician. In some embodiments, the subject's/patron's primary care physician has access to the aforementioned computer system so that the subject's/patron's condition and/or fitness or other physical progress can be monitored. For example, in some embodiments, the one or more members of the fitness facility staff may collaborate with the subject's physician in order to establish and monitor an exercise program and/or diet. The subject and/or one or more staff members may enter information about completed exercise routines, food intake, body fat percentage measurements, and the like into a central computer system located in the facility, or associated with the facility, and the subject's physician may then view those entries by accessing the system electronically using a handheld device, computer, or other computing system.

In some embodiments, the fitness facility may facilitate specialty referrals to endocrine and/or weight-loss specialists—or preventive medicine specialists, when such action would be beneficial to the scanned subject. In various embodiments, the invention teaches a method for delivering body composition analysis (e.g. body fat percentage testing) and/or bone density analysis to a subject at a fitness facility configured in any manner described above. In some embodiments, the invention includes scanning the subject's body by utilizing a DEXA scanner located at the fitness facility; obtaining results regarding the subject's body composition and/or bone density based on the DEXA scan; and providing results to the subject regarding the subject's body composition (including or limited to body fat percentage) and/or bone density.

In various embodiments, the invention teaches a method for generating revenue for a fitness facility. In certain embodiments, the method includes (1) performing one or more services that include DEXA scanning of a subject/patron at the fitness facility, (2) providing the results of the DEXA scanning to the subject/patron at the fitness facility, and (3) collecting a fee from said subject/patron for performing said DEXA scanning. In certain embodiments, the fee is charged to the subject/patron separately from a periodic membership fee paid by the subject/patron for the use of the fitness facility. In certain embodiments, the subject pays a membership fee that entitles the subject to receive at least one DEXA scan at the fitness facility, as well as the results thereof. In some embodiments, one or more of the various services available at the facility, as described above, can be sold separately. In other embodiments, one or more of the various services available at the facility are sold as part of a package of services.

In some embodiments, the revenue generating method includes establishing a membership model in which members pay a membership fee that decreases with weight loss (or gain, if the patient is under-weight) and/or bone density improvement measured by a DEXA scan, and/or body fat percentage improvement (increase or decrease depending upon the subject's individual circumstances). In this way, the revenue generating model encourages subjects/patrons to work towards beneficial results.

In some embodiments, the invention teaches a revenue generating method in which the cost of joining or maintaining a membership to a fitness facility described herein is discounted or eliminated when the subject, or the subject's insurance provider (or Medicare), pays for one or more DEXA scans performed at the fitness facility.

In various embodiments, the invention teaches a method for determining and/or modifying an exercise and/or nutritional regimen for a subject at a fitness facility. In some embodiments, the method includes utilizing a DEXA scanner located at the fitness facility to perform a DEXA scan of the subject in order to determine bone density and/or body composition of the subject (including or limited to body fat percentage), and determining and/or modifying an exercise and/or nutritional regimen for the subject based upon the results of said DEXA scan. In some embodiments, the exercise and/or nutritional regimen for the subject is determined and/or modified by a personal trainer and/or a nutritionist who are staff members of the fitness facility. In other embodiments, the exercise and/or nutritional regiment for the subject is determined and/or modified by any qualified service provider identified above.

In various embodiment, the invention teaches a fitness facility described as configured above and/or in the referenced figures.

### EXAMPLES

### Example 1

### DEXA Scanning at a Fitness Facility

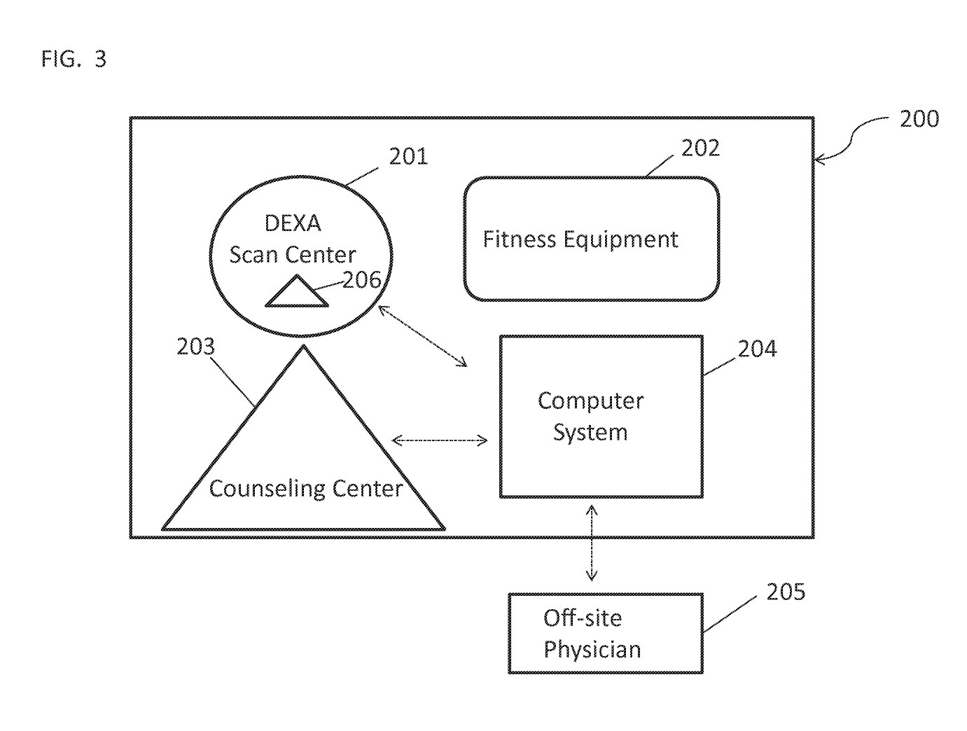

Referring to FIG. 3, a subject/patron of fitness facility 200 is scanned by a DEXA technologist using a DEXA scanner 206 located at a DEXA Scan Center 201 within fitness facility 200. After the subject/patron is scanned using the DEXA scanner 206, the subject/patron can visit an on-site Counseling Center 203 at which a certified personal trainer and/or a nutrition specialist can communicate the results of the DEXA scan to the subject/patron, as ascertained by an individual certified to read a DEXA scan. The certified personal trainer and/or nutrition expert can further interview and/or test the subject/patron and counsel the subject/patron regarding a recommended exercise routine that can be performed on Fitness Equipment 202 and/or a recommended nutrition regimen designed to accomplish the subject's health/fitness goals. The certified personal trainer and/or nutrition expert may further recommend certain on-site services that may be helpful in achieving the subject's/patron's goals (e.g. acupuncture, massage, yoga, physical therapy, health education classes, follow up DEXA testing, etc.). The subject/patron can then choose which services he or she would like to purchase and then pay for those services separately, or as part of a pre-determined bundle of services offered by the fitness facility 200.

### Example 2

### Computing Device

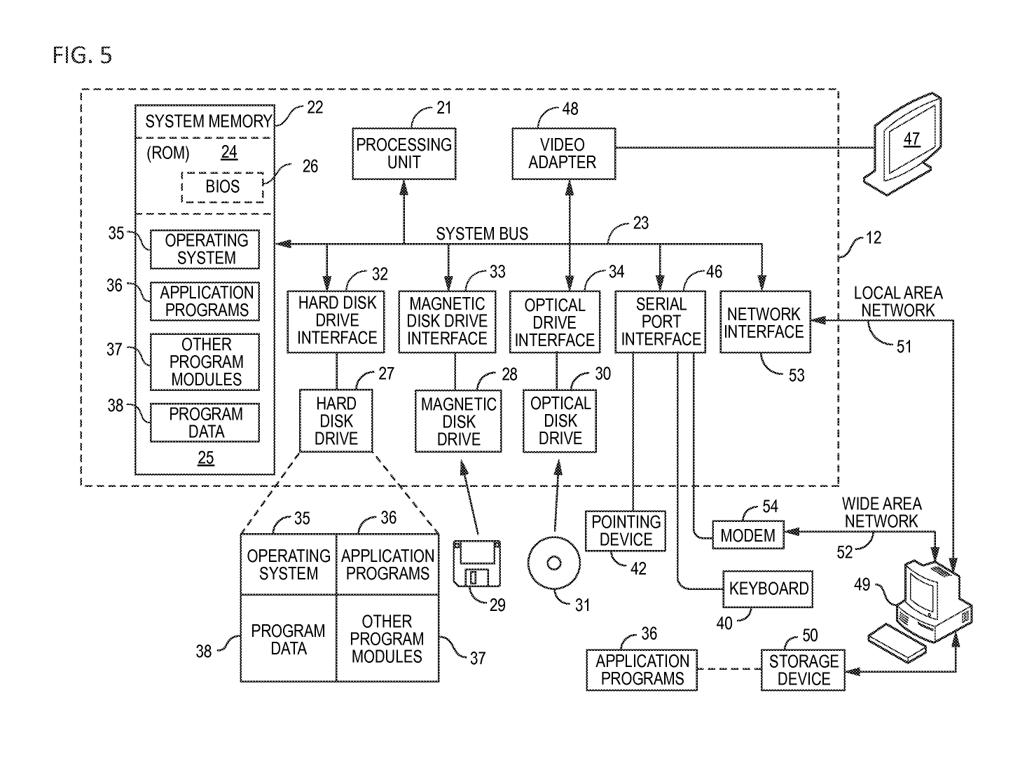

FIG. 5 is a diagram of hardware and an operating environment in conjunction with which implementations of the computing devices (e.g. 204 and 305) described herein may be practiced and utilized. The description of FIG. 5 is intended to provide a brief, general description of suitable computer hardware and a suitable computing environment in which implementations may be practiced. Although not required, implementations are described in the general context of computer-executable instructions, such as program modules, being executed by a computer, such as a personal computer. Generally, program modules include routines, programs, objects, components, data structures, etc., that perform particular tasks or implement particular abstract data types.

Moreover, those skilled in the art will appreciate that implementations may be practiced with other computer system configurations, including hand-held devices, multiprocessor systems, microprocessor-based or programmable consumer electronics, network PCs, minicomputers, mainframe computers, and the like. Implementations may also be practiced in distributed computing environments where tasks are performed by remote processing devices that are linked through a communications network. In a distributed computing environment, program modules may be located in both local and remote memory storage devices.

One of skill in the art would readily appreciate the numerous ways in which the inventive methods could be implemented through the use of cloud computing, whereby shared resources, software, and information associated with and/or generated within the presently disclosed fitness environment are provided to computers and other devices as a utility over a network, including the internet.

The exemplary hardware and operating environment of FIG. 5 includes a general-purpose computing device in the form of a computing device 12. Computing systems 204 and 305 may each be implemented using one or more computing devices like the computing device 12.

The computing device 12 includes a system memory 22, the processing unit 21, and a system bus 23 that operatively couples various system components, including the system memory 22, to the processing unit 21. There may be only one or there may be more than one processing unit 21, such that the processor of computing device 12 includes a single central-processing unit (“CPU”), or a plurality of processing units, commonly referred to as a parallel processing environment. When multiple processing units are used, the processing units may be heterogeneous. By way of a non-limiting example, such a heterogeneous processing environment may include a conventional CPU, a conventional graphics processing unit (“GPU”), a floating-point unit (“FPU”), combinations thereof, and the like. The computing device 12 may be a conventional computer, a distributed computer, or any other type of computer.

The system bus 23 may be any of several types of bus structures including a memory bus or memory controller, a peripheral bus, and a local bus using any of a variety of bus architectures. The system memory 22 may also be referred to as simply the memory, and includes read only memory (ROM) 24 and random access memory (RAM) 25. A basic input/output system (BIOS) 26, containing the basic routines that help to transfer information between elements within the computing device 12, such as during start-up, is stored in ROM 24. The computing device 12 further optionally includes a hard disk drive 27 for reading from and writing to a hard disk, not shown, a magnetic disk drive 28 for reading from or writing to a removable magnetic disk 29, and an optical disk drive 30 for reading from or writing to a removable optical disk 31 such as a CD ROM, DVD, or other optical media.

The hard disk drive 27, magnetic disk drive 28, and optical disk drive 30 are connected to the system bus 23 by a hard disk drive interface 32, a magnetic disk drive interface 33, and an optical disk drive interface 34, respectively. The drives and their associated computer-readable media provide nonvolatile storage of computer-readable instructions, data structures, program modules, and other data for the computing device 12. It should be appreciated by those skilled in the art that any type of computer-readable media which can store data that is accessible by a computer, such as magnetic cassettes, flash memory cards, solid state memory devices (“SSD”), USB drives, digital video disks, Bernoulli cartridges, random access memories (RAMs), read only memories (ROMs), and the like, may be used in the exemplary operating environment. As is apparent to those of ordinary skill in the art, the hard disk drive 27 and other forms of computer-readable media (e.g., the removable magnetic disk 29, the removable optical disk 31, flash memory cards, SSD, USB drives, and the like) accessible by the processing unit 21 may be considered components of the system memory 22.

A number of program modules may be stored on the hard disk drive 27, magnetic disk 29, optical disk 31, ROM 24, or RAM 25, including an operating system 35, one or more application programs 36, other program modules 37, and program data 38. A user may enter commands and information into the computing device 12 through input devices such as a keyboard 40 and pointing device 42. Other input devices (not shown) may include a microphone, joystick, game pad, satellite dish, scanner, touch sensitive devices (e.g., a stylus or touch pad), video camera, depth camera, or the like. These and other input devices are often connected to the processing unit 21 through a serial port interface 46 that is coupled to the system bus 23, but may be connected by other interfaces, such as a parallel port, game port, a universal serial 5 bus (USB), or a wireless interface (e.g., a Bluetooth interface). A monitor 47 or other type of display device is also connected to the system bus 23 via an interface, such as a video adapter 48. In addition to the monitor, computers typically include other peripheral output devices (not shown), such as speakers, printers, and haptic devices that provide tactile and/or other types physical feedback (e.g., a force feedback game controller).

The computing device 12 may operate in a networked environment using logical connections to one or more remote computers, such as remote computer 49. These logical connections are achieved by a communication device coupled to or a part of the computing device 12 (as the local computer). Implementations are not limited to a particular type of communications device.

The remote computer 49 may be another computer, a server, a router, a network PC, a client, a memory storage device, a peer device or other common network node, and typically includes many or all of the elements described above relative to the computing device 12. The remote computer 49 may be connected to a memory storage device 50. The logical connections depicted in FIG. 5 include a local-area network (LAN) 51 and a wide-area network (WAN) 52. Such networking environments are commonplace in offices, enterprise-wide computer networks, intranets and the Internet.

Those of ordinary skill in the art will appreciate that a LAN may be connected to a WAN via a modem using a carrier signal over a telephone network, cable network, cellular network, or power lines. Such a modem may be connected to the computing device 12 by a network interface (e.g., a serial or other type of port). Further, many laptop computers may connect to a network via a cellular data modem.

When used in a LAN-networking environment, the computing device 12 is connected to the local area network 51 through a network interface or adapter 53, which is one type of communications device. When used in a WAN networking environment, the computing device 12 typically includes a modem 54, a type of communications device, or any other type of communications device for establishing communications over the wide area network 52, such as the Internet.

The modem 54, which may be internal or external, is connected to the system bus 23 via the serial port interface 46. In a networked environment, program modules depicted relative to the personal computing device 12, or portions thereof, may be stored in the remote computer 49 and/or the remote memory storage device 50. It is appreciated that the network connections shown are exemplary and other means of and communications devices for establishing a communications link between the computers may be used.

The computing device 12 and related components have been presented herein by way of particular example and also by abstraction in order to facilitate a high-level view of the concepts disclosed. The actual technical design and implementation may vary based on particular implementation while maintaining the overall nature of the concepts disclosed.

One skilled in the art will recognize many methods and materials similar or equivalent to those described herein, which could be used in the practice of the present invention. Indeed, the present invention is in no way limited to the methods and materials described.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

What is claimed is:

1. A method for providing body fat percentage analysis and/or bone density analysis to a subject at a fitness facility, comprising:
scanning the subject's body by utilizing a dual-energy X-ray absorptiometry (DEXA) scanner located at the fitness facility;
obtaining results regarding the subject's body fat percentage and/or bone density based on the DEXA scan; and
providing results to the subject regarding the subject's body fat percentage and/or bone density.

2. The method of claim 1, wherein the results of the DEXA scan are determined at the fitness facility.

3. The method of claim 1, wherein the results of the DEXA scan are determined outside of the fitness facility.

4. The method of claim 1, wherein the fitness facility is staffed with one or more staff member trained to read and interpret the results of the DEXA scan.

5. The method of claim 1, wherein the fitness facility comprises one or more device for strength training and/or aerobic training.

6. The method of claim 1, wherein the fitness facility is staffed with one or more staff member trained to design or modify a fitness and/or nutrition and/or rehabilitation regimen for the subject at the fitness facility based partly upon the results of the DEXA scan.

7. The method of claim 1, wherein the fitness facility is staffed with an expert in nutrition selected from the group consisting of a nutritionist, a dietician, and a registered nurse.

8. The method of claim 1, wherein the fitness facility comprises a swimming pool and/or a spa and/or a sauna.

9. The method of claim 1, wherein the fitness facility is staffed with one or more certified personal trainer.

10. The method of claim 1, wherein the fitness facility is staffed with one or more medical professional selected from the group consisting of a physician, a nurse, and a dietician.

11. The method of claim 1, wherein the fitness facility comprises at least one device configured to assess cardiovascular health.

12. The method of claim 11, wherein the fitness facility is staffed with one or more staff member trained to utilize the device configured to assess cardiovascular health.

13. The method of claim 1, wherein the fitness facility is staffed with one or more staff member certified to conduct plastic surgery.

14. The method of claim 1, wherein the fitness facility comprises one or more device configured to assess pulmonary health.

15. The method of claim 14, wherein the fitness facility is staffed with one or more staff member trained to utilize the device configured to assess pulmonary health.

16. The method of claim 1, wherein the fitness facility is staffed with one or more physical therapist.

17. The method of claim 1, wherein the fitness facility is staffed with one or more staff member trained in improving or maintaining the physical and/or mental health of a subject who is from 50-100 years old.

18. The method of claim 1, wherein the subject is from 50-100 years old.

19. The method of claim 1, wherein the subject has been referred to the fitness facility by a medical professional for the purpose of recovering from an injury or illness.

20. A method for generating revenue for a fitness facility, comprising:
performing services comprising dual-energy X-ray absorptiometry (DEXA) scanning and providing the results thereof to a subject at the fitness facility; and
collecting a fee from said subject for performing said services.

21. The method of claim 20, wherein the fee for performing said services is charged separately from a periodic membership fee paid by the subject for the use of the fitness facility.

22. A method for determining and/or modifying an exercise and/or nutritional regimen for a subject at a fitness facility, comprising:
utilizing a dual-energy X-ray absorptiometry (DEXA) scanner located at the fitness facility to perform a DEXA scan of the subject in order to determine bone density and/or body fat percentage of the subject; and
determining and/or modifying an exercise and/or nutritional regimen for the subject, based at least partly upon the results of said DEXA scan.

23. The method of claim 22, wherein the exercise and/or nutritional regimen for the subject is determined and/or modified by a personal trainer and/or a nutrition expert who are staff members of the fitness facility.

24. The method of claim 23, wherein the fitness facility comprises a nutrition center that offers for sale one or more foods and/or nutritional supplements.

25. The method of claim 24, wherein one or more of the foods and/or nutritional supplements are through or known to improve bone density alone and/or in conjunction with strength training.

26. The method of claim 25, wherein one or more of the nutritional supplements is intended for consumption by a subject who is from 50-100 years of age.

27. The method of claim 23, wherein the fitness facility offers cosmetic products for sale.
